(19) United States
(12) Patent Application Publication
(10) Pub. No.: US 2025/0352709 A1
(43) Pub. Date: Nov. 20, 2025

(54) MANAGEMENT OF CONDENSED WATER IN ECMO OXYGENATOR

(71) Applicant: Maquet Critical Care AB, Solna (SE)

(72) Inventors: Mario LONCAR, Ekerö (SE); Åke LARSSON, Järfälla (SE)

(21) Appl. No.: 18/872,078

(22) PCT Filed: Jun. 5, 2023
(86) PCT No.: PCT/SE2023/050563

§ 371 Date: Dec. 5, 2024

(30) Foreign Application Priority Data
Jun. 9, 2022 (SE) 2250699-2

(51) Int. Cl. A61M 1/16

(52) U.S. Cl. CPC A61M 1/1698; A61M 2205/3334; A61M 2205/7554; A61M 2205/7563

(57) ABSTRACT

A method is for managing accumulation of condensed water in a sweep gas flow path of an oxygenator in an extracorporeal membrane oxygenator [ECMO] device. The method comprises the steps of monitoring a sweep gas flow rate and/or a sweep gas pressure within the sweep gas flow path; determining when a purge condition is met based on the monitored sweep gas flow rate and/or pressure, and performing a water purge manoeuvre when the purge condition is met. The water purge manoeuvre comprises one or more of: activating an automatic purge function of the ECMO device for automatically purging the sweep gas flow path through generation of a purge flow of sweep gas through the sweep gas flow path; causing a recommendation to activate the automated purge function of the ECMO device to be presented to an operator of the ECMO device; causing a recommendation to manually purge the sweep gas flow path to be presented to the operator of the ECMO device, and; generating an alarm indicative of accumulation of water in the sweep gas flow path.

### TECHNICAL FIELD

The present disclosure relates to the field of extracorporeal blood gas exchange and, in particular, to a system, method and computer program for managing accumulation of condensed water in a sweep gas flow path of an oxygenator in an extracorporeal membrane oxygenator (ECMO) device.

### BACKGROUND ART

When managing the treatment of a patient undergoing extracorporeal membrane oxygenator (ECMO) treatment, the oxygenator is supplied with a sweep gas flow. This flow comprises a gas mixture of one or more of oxygen, air and carbon dioxide.

As described in U.S. Pat. No. 8,585,968, when utilizing a conventional oxygenator, water vapour from the patient's blood can permeate the hollow fibre membrane and condense in the membrane's micro-pores. This condensation effectively reduces gas exchange efficiency of the oxygenator.

To remove condensed water in the oxygenator, “coughing” or “sneezing” of the oxygenator has been suggested as a method to increase the instantaneous flow across the oxygenator, thereby effecting a purge (removal of the accumulated moisture) similar to a cough or a sneeze in a patient. There are limitations to this method, however. Since the coughing of the oxygenators raises the pressure of the sweep gas compartment (i.e., the lumens of the hollow fibre), the risk of gas embolus forming in the blood and flowing back to the patient is dramatically increased.

Accordingly, when the oxygenator is being coughed, the increased pressure of the sweep gas in the hollow fibres should never exceed the pressure in the blood compartment (typically below 200 mmHg). However, since positive pressure is utilized to increase the sweep gas pressure to generate the “cough,” it is often difficult, if not impossible, to prevent the sweep gas pressure from becoming higher than the pressure in the blood compartment of the oxygenator.

Consequently, there is a need for improved management of condensation water in ECMO oxygenators.

### SUMMARY

It is an object of the present disclosure to mitigate, alleviate or eliminate one or more of the above-identified deficiencies and disadvantages in the prior art and solve at least the above mentioned problem.

According to a first aspect of the present disclosure there is provided a method for managing accumulation of condensed water in a sweep gas flow path of an oxygenator in an extracorporeal membrane oxygenator (ECMO) device. The method comprises the steps of: monitoring a sweep gas flow rate and/or a sweep gas pressure within the sweep gas flow path; determining when a purge condition is met based on the monitored sweep gas flow rate and/or pressure, and performing a water purge manoeuvre when the purge condition is met. The water purge manoeuvre comprises one or more of the following steps:
activating an automatic purge function of the ECMO device for automatically purging the sweep gas flow path through generation of a purge flow of sweep gas through the sweep gas flow path,
causing a recommendation to activate the automated purge function of the ECMO device to be presented to an operator of the ECMO device,
causing a recommendation to manually purge the sweep gas flow path to be presented to the operator of the ECMO device, and
generating an alarm indicative of accumulation of water in the sweep gas flow path.

According to some embodiments, the method comprises the steps of determining a variability of the monitored sweep gas flow rate and/or a variability of the monitored pressure indicative of change in a sweep gas flow path resistance, wherein the purge condition is a condition for the variability of sweep gas flow rate and/or the variability of the pressure.

According to some embodiments, the purge condition is met when a threshold value for a change in the monitored pressure is exceeded at a substantially constant sweep gas flow rate.

According to some embodiments, the method comprises the step of determining a sweep gas flow path resistance based on the monitored sweep gas flow rate and/or sweep gas pressure, wherein the purge condition is a condition for the sweep gas flow path resistance.

Thus, according to some embodiments, the method comprises the steps of:
determining a purge condition as a condition for a monitored sweep gas flow path resistance;
monitoring a sweep gas flow rate and a sweep gas pressure within the sweep gas flow path;
determining the monitored sweep gas flow path resistance based on the monitored sweep gas flow rate and sweep gas pressure;
determining when the purge condition is met based on the monitored sweep gas flow path resistance, and
performing a water purge manoeuvre when the purge condition is met,
wherein the water purge manoeuvre comprises:
   activating an automatic purge function of the ECMO device (5) for automatically purging the sweep gas flow path through generation of a purge flow of sweep gas through the sweep gas flow path, or
   causing a recommendation to activate the automated purge function of the ECMO device (5) to be presented to an operator of the ECMO device (5), or
   causing a recommendation to manually purge the sweep gas flow path to be presented to the operator of the ECMO device (5), or
   generating an alarm indicative of accumulation of water in the sweep gas flow path.

Whereas sweep gas flow rate and sweep gas pressure may vary over time due to manual or automatic changes in ECMO device settings, the sweep gas flow path resistance should remain substantially constant unless there is an accumulation of condensation water or other matter in the sweep gas flow path. Therefore, the use of sweep gas flow path resistance as purge condition provides for a more reliable and robust purge manoeuvre compared to purge manoeuvres that are triggered by changes in sweep gas pressure and/or flow rate.

According to some embodiments, the purge condition is a condition for a variability of the monitored sweep gas flow path resistance, wherein the method comprises the steps of determining a variability of the monitored sweep gas flow path resistance over time, and performing the water purge manoeuvre when the variability of the monitored sweep gas flow path resistance meets the purge condition.

The purge condition for the variability of the monitored sweep gas flow path resistance may, for example, be a threshold value for a change in monitored sweep gas flow resistance. In some examples, the purge condition may be a maximum threshold value for the monitored sweep gas flow path resistance. The purge condition may be considered to be met when the monitored sweep gas flow path resistance exceeds the maximum threshold value, or when the monitored sweep gas flow path resistance has exceeded the maximum threshold value for a predetermined period of time. The latter may be advantageous in situations where the sweep gas flow rate and/or the sweep gas pressure fluctuates rapidly, since such fluctuations may introduce short-term errors in the calculation of the sweep gas flow path resistance.

The purge condition may be determined based on a monitored sweep gas flow rate and a monitored sweep gas pressure monitored during a period of time constituting a time period for purge condition determination. This time period for purge condition determination is advantageously a period of time during which the sweep gas flow path can be assumed to contain a minimum of water content. For example, the time period for purge condition determination may occur immediately after start-up of the ECMO device, when hoses, membranes and gas compartments constituting the sweep gas flow path of the ECMO device are void of condensed water.

In the alternative, in some embodiments, the purge condition may be determined based on an estimated sweep gas flow path resistance, which resistance is estimated based on a configuration of the ECMO device. ECMO systems using the same or similar oxygenator and the same or similar configurations of sweep gas inlet lines and sweep gas outlet lines can be assumed to exhibit the same or similar sweep gas flow path resistances. Therefore, in some situations, it may be sufficient to set the purge condition based on a reference sweep gas flow path resistance for the particular type of ECMO system used.

According to some embodiments, the purge flow of sweep gas is at least 10 l/min.

According to another aspect of the present disclosure there is provided a computer program for preventing accumulation of condensed water in a sweep gas flow path of an oxygenator. The computer program comprising computer-readable instructions which, when executed by a control computer, causes the above described method to be performed.

According to another aspect of the present disclosure there is provided a computer program product comprising a non-transitory memory hardware device storing the above mentioned computer program.

According to another aspect of the present disclosure there is provided a system for preventing accumulation of condensed water in a sweep gas flow path of an oxygenator. The system comprises an ECMO device for extracorporeal blood gas exchange, wherein the ECMO device comprises an oxygenator including a membrane acting as a gas-liquid barrier enabling gas exchange between a bloodstream and a sweep gas flow through the oxygenator, a sweep gas flow path comprising at least a sweep gas inlet line for conveying sweep gas towards the membrane and a sweep gas outlet line for conveying sweep gas away from the membrane, at least one flow rate sensor for measuring a sweep gas flow rate within the sweep gas flow path, and/or at least one pressure sensor for measuring a sweep gas pressure within the sweep gas flow path. The system further comprises at least one control computer comprised in or coupled to the ECMO device, wherein the control computer is configured to monitor the sweep gas flow rate measured by the at least one flow rate sensor and/or the sweep gas pressure measured by the at least one pressure sensor, determine when a purge condition is met based on the monitored sweep gas flow rate and/or sweep gas pressure, and perform a water purge manoeuvre when the purge condition is met. The water purge manoeuvre comprises one or more of the following steps:
activating an automatic purge function of the ECMO device for automatically purging the sweep gas flow path through generation of a purge flow of sweep gas through the sweep gas flow path,
causing a recommendation to activate the automated purge function of the ECMO device to be presented to an operator of the ECMO device,
causing a recommendation to manually purge the sweep gas flow path to be presented to the operator of the ECMO device, and
generating an alarm indicative of accumulation of water in the sweep gas flow path.

According to some embodiments, the control computer is configured to determine a variability of the monitored sweep gas flow rate and/or a variability of the monitored pressure indicative of change in a sweep gas flow path resistance, wherein the purge condition is a condition for the variability of sweep gas flow rate and/or the variability of the pressure.

According to some embodiments, the control computer is configured to judge the purge condition to be met when a threshold value for a change in the monitored pressure is exceeded at a substantially constant sweep gas flow rate.

According to some embodiments, the control computer is configured to determine a sweep gas flow path resistance based on the monitored sweep gas flow rate and/or sweep gas pressure, wherein the purge condition is a condition for the sweep gas flow path resistance.

Thus, according to some embodiments, the system comprises:
an extracorporeal membrane oxygenator [ECMO] device (5) for extracorporeal blood gas exchange, comprising:
   an oxygenator (21) including a membrane (23) acting as a gas-liquid barrier enabling gas exchange between a bloodstream and a sweep gas flow through the oxygenator (21),
   a sweep gas flow path comprising at least a sweep gas inlet line (33a) for conveying sweep gas towards the membrane (23), and a sweep gas outlet line (33b) for conveying sweep gas away from the membrane (23),
   at least one flow rate sensor (29a, 31a) for measuring a sweep gas flow rate within the sweep gas flow path and/or at least one pressure sensor (29d) for measuring a sweep gas pressure within the sweep gas flow path, and
at least one control computer (27) comprised in or coupled to the ECMO device (5), wherein the control computer (27) is configured to:
   determine a purge condition as a condition for a monitored sweep gas flow path resistance;
   monitor the sweep gas flow rate measured by the at least one flow rate sensor (29a, 31a), and the sweep gas pressure measured by the at least one pressure sensor (29d);
   determine the monitored sweep gas flow path resistance based on the monitored sweep gas flow rate and sweep gas pressure;
   determine when the purge condition is met based on the monitored sweep gas flow path resistance, and
   perform a water purge manoeuvre when the purge condition is met,
wherein the water purge manoeuvre comprises:
   activating an automatic purge function of the ECMO device (5) for automatically purging the sweep gas flow path through generation of a purge flow of sweep gas through the sweep gas flow path, or
   causing a recommendation to activate the automated purge function of the ECMO device (5) to be presented to an operator of the ECMO device (5), or
   causing a recommendation to manually purge the sweep gas flow path to be presented to the operator of the ECMO device (5), or
   generating an alarm indicative of accumulation of water in the sweep gas flow path.

According to some embodiments, the purge condition is a condition for a variability of the monitored sweep gas flow path resistance, wherein the control computer is configured to determine the variability of the monitored sweep gas flow path resistance over time, and perform the water purge manoeuvre when the variability of the monitored sweep gas flow path resistance meets the purge condition.

According to some embodiments, the control computer is configured to determine the purge condition based on a monitored sweep gas flow rate and a monitored sweep gas pressure, monitored during a period of time constituting a time period for purge condition determination.

As discussed above, the purge condition for the variability of the monitored sweep gas flow path resistance may, for example, be a threshold value for a change in monitored sweep gas flow resistance. In some examples, the purge condition may be a maximum threshold value for the monitored sweep gas flow path resistance. The control computer may be configured to determine that the purge condition is met when the monitored sweep gas flow path resistance exceeds the maximum threshold value, or when the monitored sweep gas flow path resistance has exceeded the maximum threshold value for a predetermined period of time.

The control computer may be configured to determine the purge condition based on a monitored sweep gas flow rate and a monitored sweep gas pressure monitored during a period of time constituting a time period for purge condition determination. This time period for purge condition determination is advantageously a period of time during which the sweep gas flow path can be assumed to contain a minimum of water content. For example, the time period for purge condition determination may occur immediately after start-up of the ECMO device, when hoses, membranes and gas compartments constituting the sweep gas flow path of the ECMO device are void of condensed water.

In the alternative, in some embodiments, the control computer may be configured to determine the purge condition based on an estimated sweep gas flow path resistance, which resistance is estimated based on a configuration of the ECMO device.

According to some embodiments, the ECMO device further comprises a sweep gas generator for generating a sweep gas flow through the sweep gas flow path, the control computer being configured to control the sweep gas generator to generate the purge flow of sweep gas upon activation of the automatic purge function.

According to some embodiments, the purge flow of sweep gas is at least 10 l/min.

According to some embodiments, the control computer is comprised in the ECMO device. In other embodiments, the control computer is a control computer of the sweep gas flow generator. In yet other embodiments, the control computer is a control computer of a separate purge device coupled to the sweep gas flow generator. In this case, when the purge condition is met, the purge device may perform the water purge manoeuvre by signalling to the sweep gas flow generator that purging should be performed, thereby activating an automatic purge function of the ECMO device

Consequently, it should be realized that the above described computer program for preventing accumulation of condensed water in a sweep gas flow path of an oxygenator may reside in any of a control computer of an ECMO device, a control computer of a sweep gas flow generator, or a control computer of a purge device coupled to the sweep gas flow generator.

Thus, according to another aspect of the present disclosure, there is provided a device comprising a control computer including at least one processor and a data storage medium, such as a non-transitory memory hardware device, storing the above described computer program, wherein the control computer, upon execution of the computer program by the at least one processor, causes the above described method to be performed. As is clear from the foregoing description, the device may be a sweep gas flow generator for generating a flow of sweep gas through a sweep gas flow path of an ECMO device, or a purge device coupled to such a sweep gas flow generator.

More advantageous features of the method, computer program and system of the present disclosure will be described in the detailed description following hereinafter.

### BRIEF DESCRIPTIONS OF THE DRAWINGS

The present invention will become more fully understood from the detailed description provided hereinafter and the accompanying drawings, which are given by way of non-limiting illustration only. In the different drawings, same reference numerals correspond to the same element.

FIG. 1 illustrates an exemplary non-limiting embodiment of a combined ECMO and ventilator system for extracorporeal removal of CO2 from the blood of a patient undergoing mechanical ventilation.

FIG. 2 illustrates an exemplary non-limiting embodiment of an ECMO device, which ECMO device incorporates functionality for management of condensed water in a sweep gas flow path of the ECMO device.

### DETAILED DESCRIPTION

The present disclosure will now be described with reference to the accompanying drawings, in which preferred example embodiments of the disclosure are shown. The disclosure may, however, be embodied in other forms and should not be construed as limited to the herein disclosed embodiments. The disclosed embodiments are provided to fully convey the scope of the disclosure to the skilled person.

Although the present disclosure relates to a function for management of condensed water in an oxygenator of an extracorporeal membrane oxygenator (ECMO) device, the function will hereinafter be described in the context of a combined system, herein referred to as an ECMO-vent system, comprising both a mechanical ventilator and an ECMO device for extracorporeal removal of CO2 from the blood of the patient 3. However, it should be realized that the principles of condensation water management in an ECMO device, as disclosed herein, are in no way limited to this particular system setup or the presence of a mechanical ventilator.

ECMO (extracorporeal membrane oxygenation) is one of several terms used for extracorporeal blood gas exchange where blood is pumped outside the body of a treated patient to a device, sometimes referred to as a heart-lung machine, which removes CO2 and sends oxygen-enriched blood back to the patient. Other terms that are frequently used in the art for the same or similar treatments are ECLA (extracorporeal lung assist), ECCO2R (extracorporeal CO2 removal), ECLS (extracorporeal life support) and ECGE (extracorporeal membrane gas-exchange), all of which are encompassed by the term ECMO as used herein.

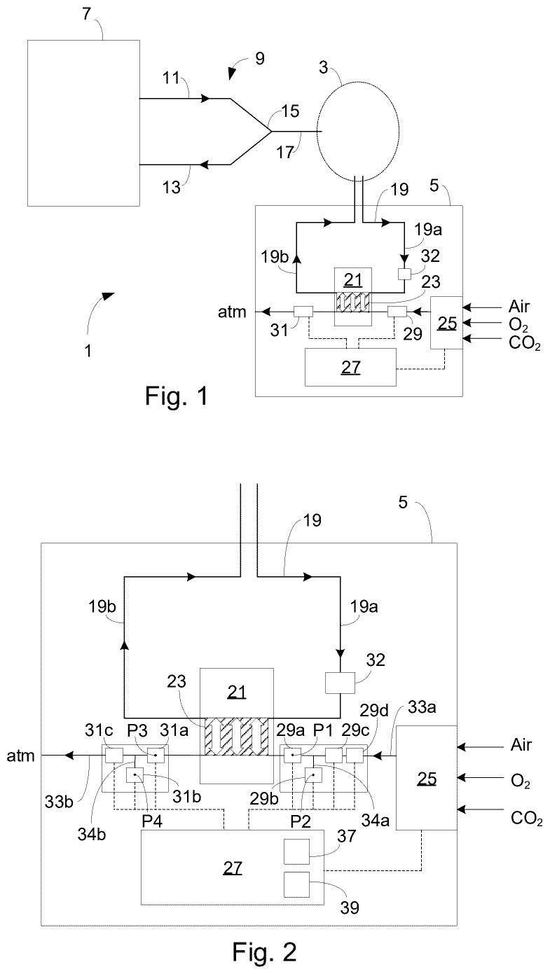

FIG. 1 illustrates an ECMO-vent system 1 comprising a device 5, herein referred to as an ECMO device, for extracorporeal removal of CO2 from the blood of the patient 3, and a mechanical ventilator 7 for mechanically ventilating the patient 3 through the supply of breathing gas to the lungs of the patient.

The ventilator 7 comprises or is connected to a source of pressurised breathing gas (not shown), which breathing gas is supplied to the patient 3 via a patient circuit 9. In this example, the patient circuit 9 comprises an inspiratory line 11 for conveying a flow of breathing gas to the patient 3, and an expiratory line 13 for conveying a flow of exhalation gas exhaled by the patient away from the patient. The inspiratory line 11 and the expiratory line 13 are connected to each other via a so called Y-piece 15 which, in turn, is connected to the patient 3 via a common line 17.

The ECMO device 5 is configured to provide ECMO treatment to the patient 3 by generating an extracorporeal flow of blood from the patient 3, oxygenating the blood through extracorporeal blood gas exchange in which CO2 is removed from, and oxygen (O2) added to, the extracorporeal blood flow, and returning the oxygen-enriched blood to the patient 3.

To generate the flow of blood to and from the patient 3, the ECMO device 5 may comprise a blood flow generator (not shown), typically in form of one or several roller, turbine and/or centrifugal pumps. The blood flow generator generates a flow of blood through a tubing system forming a blood flow channel 19 of the ECMO device 5, where parts of the channel may be heated to maintain a desired temperature of the blood when returned to the patient 3.

The blood gas exchange, including blood oxygenation and CO2 removal, takes place in a membrane oxygenator 21 of the ECMO device 5, in which an oxygen-containing sweep gas flow interacts with the blood in the blood flow channel 19 via a membrane 23 of the oxygenator 21. The membrane 23 acts as a gas-liquid barrier enabling transfer of CO2 and O2 content between the bloodstream flowing through the oxygenator 21 on a liquid-side of the membrane 23 and the sweep gas flow flowing through the oxygenator 21 on a gas-side of the membrane 23.

The sweep gas flow is generated by a sweep gas generator 25 connected to one or more sweep gas sources, typically including one or both of an oxygen source and a source of compressed air. According to the principles of the present disclosure, the sweep gas generator 25 is further connected to a CO2 source in order to control the degree of CO2 removal over the oxygenator 21 through addition of CO2 to the sweep gas flow. The sweep gas generator 25 is configured to deliver a controllable sweep gas composition to the oxygenator 21 at a controllable sweep gas flow rate. In clinical practice, a sweep gas flow generator is often referred to as a sweep gas mixer, a sweep gas blender, a gas blender or an electronic gas blender (EGB).

The composition and, optionally, the flow rate of the sweep gas generated by the sweep gas generator 25 may be automatically controlled by a controller or control computer 27 of the ECMO device 5 based on set target values and sensor data obtained by various sensors 29, 31 of the ECMO device 5. In particular, the control computer 27 of the ECMO device 5 may be configured to automatically control an addition of CO2 to a sweep gas flow comprising any or both of oxygen and air, based on a set target for a measure of CO2 removal by the oxygenator 21.

Hereinafter, the sweep gas flow upstream of the oxygenator 21 (i.e., before the oxygenator from the sweep gas' point of view) will be referred to as an input sweep gas flow or a pre-oxygenator sweep gas flow, and the sweep gas flow downstream of the oxygenator 21 (i.e., after the oxygenator from the sweep gas' point of view) will be referred to as an output sweep gas flow or a post-oxygenator sweep gas flow. The input sweep gas flow flows from the sweep gas generator 25 to the oxygenator 21 via a sweep gas inlet line 33a of the ECMO device 5, and the output sweep gas flow flows from the oxygenator 21 to atmosphere or an evacuation or recirculation system via a sweep gas outlet line 33b. In most configurations, ECMO systems are open systems, meaning that the post oxygenator sweep gas flow is allowed to escape into the ambient. In some cases, especially when anesthetic agents are added to the sweep gas flow, a closed or semi closed (sweep) gas control system can be envisioned, similar to gas control systems often used in anesthesia machines.

Likewise, the bloodstream upstream of the oxygenator 21 (i.e., before the oxygenator from the bloodstream's point of view) may hereinafter be referred to as an input bloodstream or pre-oxygenator bloodstream, and the bloodstream downstream of the oxygenator 21 (i.e., after the oxygenator from the bloodstream's point of view) may be referred to as an output bloodstream or post-oxygenator bloodstream. The input bloodstream flows from the patient 3 to the oxygenator 21 via a bloodstream inlet line 19a of the ECMO device 5, and the output bloodstream flows from the oxygenator 21 and back to the patient 3 via a bloodstream outlet line 19b of the ECMO device 5.

With reference now made to FIG. 2, the sensors 29, 31 of the ECMO device 5 may comprise:
a pre-oxygenator flow rate sensor 29a for measuring a flow rate of the input sweep gas flow, Vᵢₙ. The pre-oxygenator flow rate sensor 29a is a mainstream flow sensor, meaning that it is configured to measure the flow rate of the sweep gas flowing in the sweep gas inlet line 33a. The pre-oxygenator flow measurements obtained by the pre-oxygenator flow rate sensor 29a take place at a pre-oxygenator point of flow measurement denoted P1 in the sweep gas inlet line 33a.
a pre-oxygenator gas analyser 29b for measuring a fraction of at least CO2 in the input sweep gas flow, FCO2ᵢₙ. The pre-oxygenator gas analyser 29b may also be configured to measure a fraction of one or more additional gases selected from the group consisting of oxygen (O2), nitrogen gas (N2), and anaesthetic agents. In this exemplary embodiment, the pre-oxygenator gas analyser 29b is a so called sidestream gas analyser that is configured to withdraw sweep gas samples from the sweep gas inlet line 33a, and measure the fraction of CO2 and, optionally, the fraction of the at least one additional gas in the sweep gas samples at a pre-oxygenator point of CO2 measurement denoted P2. The pre-oxygenator point of CO2 measurement P2 is separated in distance from the point of pre-oxygenator sweep gas flow rate measurements, P1, at least by the length of a pre-oxygenator sample line 34a. In other embodiments, the pre-oxygenator gas analyser 29b may be a so called mainstream gas analyser that is configured to measure the fraction of CO2 and, optionally, the fraction of the at least one additional gas within the sweep gas inlet line 33a. In some embodiments, the pre-oxygenator gas analyser comprises at least a CO2 sensor and an O2 sensor for measuring a fraction of CO2 and O2, respectively, in the sweep gas samples. In some embodiments, the CO2 sensor is a non-dispersive infrared (NDIR) CO2 sensor. In some embodiments, the O2 sensor is a paramagnetic or electrochemical O2 sensor.
a pre-oxygenator temperature sensor 29c for measuring a temperature of the input sweep gas, T_{in,gas}.
a pressure sensor 29d for measuring a sweep gas circuit pressure, P_{gas}, substantially corresponding to the sweep gas pressure in the gas inlet line 33a.
a post-oxygenator flow rate sensor 31a for measuring a flow rate of the output sweep gas flow, Vₒᵤₜ. The post-oxygenator flow rate sensor 31a is a mainstream flow sensor, meaning that it is configured to measure the flow rate of the sweep gas flowing in the sweep gas outlet line 33b. The post-oxygenator flow measurements obtained by the post-oxygenator flow rate sensor 31a take place at a post-oxygenator point of flow measurement denoted P3 in the sweep gas outlet line 33b.
a post-oxygenator gas analyser 31b for measuring a fraction of at least CO2ᵢₙ the output sweep gas flow, FCO2ₒᵤₜ. The post-oxygenator gas analyser 31b may also be configured to measure a fraction of one or more additional gases selected from the group consisting of oxygen (O2), nitrogen gas (N2), and anaesthetic agents. In this exemplary embodiment, the post-oxygenator gas analyser 31b is a so called sidestream gas analyser that is configured to withdraw sweep gas samples from the sweep gas outlet line 33b, and measure the fraction of CO2 and, optionally, the fraction of the at least one additional gas in the sweep gas samples at a post-oxygenator point of CO2 measurement denoted P4. The post-oxygenator point of CO2 measurement P4 is separated in distance from the point of post-oxygenator sweep gas flow rate measurements, P3, at least by the length of a post-oxygenator sample line 34b. In other embodiments, the post-oxygenator gas analyser 31b may be a so called mainstream gas analyser that is configured to measure the fraction of CO2 and, optionally, the fraction of the at least one additional gas within the sweep gas outlet line 33b. In some embodiments, the post-oxygenator gas analyser comprises at least a CO2 sensor and an O2 sensor for measuring a fraction of CO2 and O2, respectively, in the sweep gas samples. In some embodiments, the CO2 sensor is an IR spectrometer for IR spectroscopy, such as IR absorption spectroscopy. In some embodiments, the O2 sensor is a paramagnetic O2 sensor.
a post-oxygenator temperature sensor 31c for measuring a temperature of the output sweep gas.

In some embodiments, the ECMO device 5 may further comprise or be connected to a pre-oxygenator blood gas analyser 32 for measuring a partial pressure of at least CO2ᵢₙ the input bloodstream, PCO2ᵢₙ. The pre-oxygenator blood gas analyser 32 may also be configured to measure a partial pressure of O2ᵢₙ the input bloodstream, PO2ᵢₙ. The pre-oxygenator blood gas analyser 32 may also be configured to measure a haemoglobin content of the input bloodstream, Hbᵢₙ. In some embodiments, the blood gas analyser 32 is not incorporated into the ECMO device 5 but arranged to form part of another medical device that is connected to the ECMO device 5 in order for the ECMO device 5 to receive measurements obtained by the blood gas analyser. For example, the blood gas analyser may form part of a stand-alone blood gas analyser unit, often referred to as a BGA, commonly used for intermittent blood gas analysis during ECMO treatments.

In accordance with the principles of the present disclosure, the ECMO device 5 includes functionality for managing accumulation of condensed water in the sweep gas flow path of the oxygenator 21. The control computer 27 is configured to monitor the sweep gas flow rate measured by the at least one flow rate sensor 29a, 31a, and/or the sweep gas pressure measured by the at least one pressure sensor 29d, and to determine when a purge condition is met based on the monitored sweep gas flow rate and/or sweep gas pressure. If and when the purge condition is met, the control computer 27 is configured to perform a water purge manoeuvre by:
activating an automatic purge function of the ECMO device 5 for automatically purging the sweep gas flow path through generation of a purge flow of sweep gas through the sweep gas flow path,
causing a recommendation to activate the automated purge function of the ECMO device 5 to be presented to an operator of the ECMO device 5,
causing a recommendation to manually purge the sweep gas flow path to be presented to the operator of the ECMO device 5, and/or
generating an alarm indicative of accumulation of water in the sweep gas flow path.

The proposed functionality thus relies on a pressure and/or flow criteria controlled water purge manoeuvre, which manoeuvre may involve activation of an automatic purge function of the ECMO device 5 or cause other actions that assist the user in the management of accumulation of condensed water in the sweep gas flow path to be performed.

That the water purge manoeuvre is automatically performed when the pressure and/or flow based criteria is met serves to ensure that the water purge manoeuvre is performed only when there is a real need for purging the sweep gas flow channel. It also allows for early detection of reduced gas exchange efficiency of the oxygenator 21 since deterioration of oxygenator performance typically introduces variations in sweep gas flow rate and/or pressure.

The control computer 27 may be configured to determine a variability of the monitored sweep gas flow rate and/or a variability of the monitored pressure, indicative of change in a sweep gas flow path resistance, and the purge condition may be a condition for the variability of sweep gas flow rate and/or the variability of the pressure. This way, the purge manoeuvre can be activated in response to changes in sweep gas flow resistance caused by the accumulation of condensed water in the gas flow path.

The control computer 27 may be configured to judge the purge condition to be met when a threshold value for a change in the monitored pressure is exceeded at a substantially constant sweep gas flow rate. This is advantageous in that the pressure drop over the oxygenator 21 at constant flow rate is a reliable and robust measure of changes in sweep gas flow path resistance.

In some embodiments, the control computer 27 may be configured to determine an actual sweep gas flow path resistance based on the monitored sweep gas flow rate and the monitored sweep gas pressure, and the purge condition may be a condition for actual sweep gas flow path resistance.

The control computer 27 may further be configured to control the sweep gas generator 25 to generate the purge flow of sweep gas upon activation of the automatic purge function. For neonatal patients a smaller purge may be desired but, for adult patients, the purge flow of sweep gas should preferably be at least 10 l/min.

According to another aspect of the present disclosure, there is provided a method for managing accumulation of condensed water in a sweep gas flow path of an oxygenator 21 in an ECMO device 5. The method is typically a computer-implemented method that is performed upon execution of a computer program by the at least one processor 37 of the control computer 27. The computer program(s) comprises computer-readable instructions that may be stored in a storage medium, such as the non-transitory hardware memory device 39 of the control computer 27.

The method may comprise the steps of monitoring a sweep gas flow rate and/or a sweep gas pressure within the sweep gas flow path, determining when a purge condition is met based on the monitored sweep gas flow rate and/or pressure, and performing a water purge manoeuvre when the purge condition is met. The water purge manoeuvre comprises one or more of: activating an automatic purge function of the ECMO device 5 for automatically purging the sweep gas flow path through generation of a purge flow of sweep gas through the sweep gas flow path; causing a recommendation to activate the automated purge function of the ECMO device 5 to be presented to an operator of the ECMO device 5; causing a recommendation to manually purge the sweep gas flow path to be presented to the operator of the ECMO device 5, and; generating an alarm indicative of accumulation of water in the sweep gas flow path.

The method may further comprise the steps of determining a variability of the monitored sweep gas flow rate and/or a variability of the monitored pressure indicative of change in a sweep gas flow path resistance, wherein the purge condition is a condition for the variability of sweep gas flow rate and/or the variability of the pressure. In some embodiments, the purge condition is judged to be met when a threshold value for a change in the monitored pressure is exceeded at a substantially constant sweep gas flow rate.

In some embodiments, the method comprises the step of determining a sweep gas flow path resistance based on the monitored sweep gas flow rate and the sweep gas pressure, whereby the purge condition may be a condition for the sweep gas flow path resistance.

1-21. (canceled)

22. A method for managing accumulation of condensed water in a sweep gas flow path of an oxygenator in an extracorporeal membrane oxygenator [ECMO] device, comprising the steps of:
determining a purge condition as a condition for a monitored sweep gas flow path resistance;
monitoring a sweep gas flow rate and a sweep gas pressure within the sweep gas flow path;
determining the monitored sweep gas flow path resistance based on the monitored sweep gas flow rate and sweep gas pressure;
determining when the purge condition is met based on the monitored sweep gas flow path resistance, and
performing a water purge manoeuvre when the purge condition is met,
wherein the water purge manoeuvre comprises:
activating an automatic purge function of the ECMO device configured to automatically purge the sweep gas flow path through generation of a purge flow of sweep gas through the sweep gas flow path, or
causing a recommendation to activate the automated purge function of the ECMO device to be presented to an operator of the ECMO device, or
causing a recommendation to manually purge the sweep gas flow path to be presented to the operator of the ECMO device, or
generating an alarm indicative of accumulation of water in the sweep gas flow path.

23. The method of claim 22, wherein the purge condition is a condition for a variability of the monitored sweep gas flow path resistance, the method further comprising the steps of:
determining a variability of the monitored sweep gas flow path resistance over time, and
performing the water purge manoeuvre when the variability of the monitored sweep gas flow path resistance meets the purge condition.

24. The method of claim 22, wherein the purge condition is determined based on a monitored sweep gas flow rate and a monitored sweep gas pressure, monitored during a period of time constituting a time period for purge condition determination.

25. The method of claim 24, wherein the time period for purge condition determination is a period of time during which the sweep gas flow path can be assumed to contain a minimum of water content.

26. The method of claim 25, wherein the time period for purge condition determination is a period of time occurring substantially immediately after start-up of the ECMO device.

27. The method of claim 22, wherein the purge condition is determined based on an estimated sweep gas flow path resistance, estimated based on a configuration of the ECMO device.

28. The method of claim 22, wherein the purge flow of sweep gas is at least 10 l/min.

29. A computer program for managing accumulation of condensed water in a sweep gas flow path of an oxygenator, the computer program comprising computer-readable instructions which, when executed by a control computer, causes the method of claim 22 to be performed.

30. A computer program product comprising a non-transitory memory hardware device storing a computer program for preventing accumulation of condensed water in a sweep gas flow path of an oxygenator, the computer program comprising computer-readable instructions which, when executed by a control computer, causes the method of claim 22 to be performed.

31. A system for managing accumulation of condensed water in a sweep gas flow path of an oxygenator, comprising:
an extracorporeal membrane oxygenator [ECMO] device configured for extracorporeal blood gas exchange, comprising:
an oxygenator including a membrane acting as a gas-liquid barrier enabling gas exchange between a bloodstream and a sweep gas flow through the oxygenator,
a sweep gas flow path comprising at least a sweep gas inlet line configured to convey sweep gas towards the membrane, and a sweep gas outlet line configured to convey sweep gas away from the membrane, and
at least one flow rate sensor configured to measure a sweep gas flow rate within the sweep gas flow path and at least one pressure sensor configured to measure a sweep gas pressure within the sweep gas flow path; and
at least one control computer comprised in or coupled to the ECMO device,
wherein the control computer is configured to:
determine a purge condition as a condition for a monitored sweep gas flow path resistance;
monitor the sweep gas flow rate measured by the at least one flow rate sensor, and the sweep gas pressure measured by the at least one pressure sensor;
determine the monitored sweep gas flow path resistance based on the monitored sweep gas flow rate and sweep gas pressure;
determine when the purge condition is met based on the monitored sweep gas flow path resistance; and
perform a water purge manoeuvre when the purge condition is met,
wherein the water purge manoeuvre comprises:
activating an automatic purge function of the ECMO device configured to automatically purge the sweep gas flow path through generation of a purge flow of sweep gas through the sweep gas flow path, or
causing a recommendation to activate the automated purge function of the ECMO device to be presented to an operator of the ECMO device, or
causing a recommendation to manually purge the sweep gas flow path to be presented to the operator of the ECMO device, or
generating an alarm indicative of accumulation of water in the sweep gas flow path.

32. The system of claim 31, wherein the purge condition is a condition for a variability of the monitored sweep gas flow path resistance, the control computer being configured to:
determine the variability of the monitored sweep gas flow path resistance over time, and
perform the water purge manoeuvre when the variability of the monitored sweep gas flow path resistance meets the purge condition.

33. The system of claim 32, wherein the control computer is configured to determine the purge condition based on a monitored sweep gas flow rate and a monitored sweep gas pressure, monitored during a period of time constituting a time period for purge condition determination.

34. The system of claim 33, wherein the time period for purge condition determination is a period of time during which the sweep gas flow path can be assumed to contain a minimum of water content.

35. The system of claim 34, wherein the time period for purge condition determination is a period of time occurring substantially immediately after start-up of the ECMO device.

36. The system of claim 31, wherein the control computer is configured to determine the purge condition based on an estimated sweep gas flow path resistance, estimated based on a configuration of the ECMO device.

37. The system of claim 31, wherein the ECMO device further comprises a sweep gas generator configured to generate a sweep gas flow through the sweep gas flow path, the control computer being configured to control the sweep gas generator to generate the purge flow of sweep gas upon activation of the automatic purge function.

38. The system of claim 31, wherein the purge flow of sweep gas is at least 10 l/min.

39. A device comprising a control computer including at least one processor and a data storage medium storing the computer program of claim 29.

40. The device of claim 39, wherein the device is a sweep gas flow generator configured to generate a flow of sweep gas through a sweep gas flow path of an ECMO device.

41. The device of claim 39, wherein the device is a purge device coupled to a sweep gas flow generator configured to generate a flow of sweep gas through a sweep gas flow path of an ECMO device.

42. The device of claim 39, wherein the computer program, when executed by the at least one processor, causes the device to activate an automatic purge function of the ECMO device configured to automatically purge the sweep gas flow path through generation of a purge flow of sweep gas through the sweep gas flow path.
